(19) United States
(12) Patent Application Publication
(10) Pub. No.: US 2025/0177708 A1
(43) Pub. Date: Jun. 5, 2025

(54) SYSTEMS AND METHODS FOR MEDICAL DIAGNOSTICS AND MEDICATION DELIVERY

(71) Applicant: Zorday IP, LLC, Mayfield Village, OH (US)

(72) Inventor: Brent M. Skoda, Mayfield Village, OH (US)

(73) Assignee: Zorday IP, LLC, Mayfield Village, OH (US)

(21) Appl. No.: 18/905,999

(22) Filed: Oct. 3, 2024

Related U.S. Application Data
(63) Continuation of application No. 18/349,018, filed on Jul. 7, 2023, now abandoned.
Application No. 18/349,018 is a continuation of application No. 17/731,902, filed on Apr. 28, 2022, now abandoned.
Application No. 17/731,902 is a continuation of application No. 16/258,533, filed on Jan. 25, 2019, now abandoned.
Application No. 16/258,533 is a continuation of application No. 15/144,036, filed on May 2, 2016, now abandoned.
(60) Provisional application No. 62/155,863, filed on May 1, 2015.

(51) Int. Cl. A61M 31/00; A61B 5/00; A61B 5/11; A61C 19/06; A61M 5/142; A61M 5/20; A61M 11/04; A63B 71/10; B60R 21/01; G16H 20/10; G16H 20/30; G16H 40/63; G16H 40/67

(52) U.S. Cl. CPC A61M 31/00; A61B 5/0022; A61B 5/11; A61B 5/4064; A61C 19/06; A61M 11/042; A63B 71/10; G16H 20/10; G16H 20/30; G16H 40/63; G16H 40/67; A61C 19/063; A61M 5/14244; A61M 5/20; A61M 2205/3327; A61M 2205/3584; A61M 2205/3592; A61M 2205/502; A61M 2205/52; A61M 2209/088; A61M 2210/0625; B60R 2021/01211; B60R 2021/01252; Y02A 90/10

(57) ABSTRACT

The present disclosure relates to a medical diagnostic and delivery device that includes a mouthguard, a dental brace or a denture. The device can be positioned within a mouth or a buccal cavity of a user. A housing of the device can be shaped to be removably secured within the buccal cavity of the user, for example, include grooves shaped and sized to match a dental bite of the user. The device includes a cartridge containing one more substances or medications configured to be delivered to a user in the form a liquid, particles, or fine mist. Any suitable first air medication can be contained within the cartridge such as epinephrine, blood thinners, painkillers, vitamins or any other suitable drugs for limiting the extent of injury.

### CROSS-REFERENCE TO RELATED PATENT APPLICATIONS

The present application is a continuation of and claims the benefit of and priority to U.S. application Ser. No. 18/349,018, titled “SYSTEMS AND METHODS FOR MEDICAL DIAGNOSTICS AND MEDICATION DELIVERY” and filed Jul. 7, 2023, which is a continuation of and claims the benefit of and priority to U.S. application Ser. No. 17/731,902, titled “SYSTEMS AND METHODS FOR MEDICAL DIAGNOSTICS AND MEDICATION DELIVERY” and filed Apr. 28, 2022, which is a continuation of and claims the benefit of and priority to U.S. application Ser. No. 16/258,533, titled “SYSTEMS AND METHODS FOR MEDICAL DIAGNOSTICS AND MEDICATION DELIVERY” and filed Jan. 25, 2019, which is a continuation of and claims the benefit of and priority to U.S. application Ser. No. 15/144,036, titled “SYSTEMS AND METHODS FOR MEDICAL DIAGNOSTICS AND MEDICATION DELIVERY” and filed May 2, 2016, which, claims the benefit of and priority to U.S. Provisional Application No. 62/155,863, titled “SYSTEMS AND METHODS FOR MEDICAL DIAGNOSTICS AND MEDICATION DELIVERY” and filed May 1, 2015, the entire contents of which are hereby incorporated by reference.

### TECHNICAL FIELD

The present application relates generally to systems and methods for medical diagnostics and medication delivery.

### BACKGROUND

A central theme in medical diagnostics is the ability to diagnose one or more medical conditions at the point of care as well as deliver medication on site. Particular in trauma injuries such as traffic accidents, sports injuries such as, head injuries suffered during football, ice hockey, skiing, cycling, cricket, hockey, lacrosse, construction injuries or any other trauma injury, the mitigation of injury impact and limiting the extent of delivery depends on timely first aid delivered to the patient. For example, the first few seconds post head or spinal cord injury suffered during football (e.g., injuries causing a concussion) are critical in preventing the spread of the injury (e.g., due to ischemia or oxygen loss to brain). If first aid such as a therapeutic dose of a medication is administered to the player within this time frame, this can limit the spread and extent of injury, as well as reduce recovery time.

Timely first aid is also critical in patients suffering non-traumatic medical emergency, such as an epileptic seizure, acute glucose shortage, exposure to toxins (e.g., air borne or blood borne pathogens or chemicals), etc. Timely delivery of a therapeutic dose to a user, for example, a hospital or emergency aid worker such as a doctor, nurse or volunteer working in an environment susceptible to toxin exposure (e.g., working in an Ebola virus zone) can limit the spread of the disease or otherwise prevent incapacitation of the user.

### SUMMARY OF THE INVENTION

Embodiments described herein relate generally to medical diagnosis and delivery devices, and in particular to wearable or portable vapor and/or liquid delivery devices for providing one or more medication delivery applications.

In some embodiments, a medical diagnostic and delivery device includes a protective head gear. The device can be worn over a head and face of a user and can include a football helmet, an ice-hockey helmet, a cricket helmet, a hockey mask, a baseball helmet, a baseball referee mask, a ski helmet, a motorcycle helmet, a race car driver helmet, a construction worker helmet, or any other protective head gear. The device includes a cartridge containing one more substances or medications configured to be delivered to a user in the form of a vapor, liquid, particles, or fine mist. Any suitable first aid medication can be contained within the cartridge such as epinephrine, blood thinners (e.g., heparin, aspirin), painkillers (e.g., Tylenol®, non-steroidal anti-inflammatory drugs (NSAIDS), codine, morphine), b-vitamins or any other suitable drugs for limiting the extent of injury. In particular embodiments, the device can include a heating element to vaporize the substance and deliver the vapor to a user. In various embodiments, the device includes an injector positioned within a housing of the device. The injector can insert a needle in fluid communication with the cartridge into the user to deliver the medication intramuscularly or intravenously. The device includes a sensor, for example, an accelerometer to sense, detect or otherwise determine an impact on the head and/or any other body part of the user. The sensor can also include a gyrometer, inclinometer, or any other sensor that can detect a change in position, speed, orientation, or movement. The device also includes electronic circuitry that can include one or more of a power source, a specially programmed processor, a memory, a speaker, microphone and a display. The specially programmed processor may be configured to execute instructions for analyzing signals provided by the sensor and determining if the impact is beyond a critical or otherwise predetermined threshold. In various embodiments, in response to the impact exceeding the predetermined threshold, the processor commands the vaporizer to vaporize the medication contained within the cartridge so that the medication is delivered to the user. In other embodiments, the processor commands the injector to insert the needle into the user to deliver the medication. The electronic circuitry can also include communication and/or location devices such as Bluetooth®, Wi-Fi, RFID, cellular transceiver, and/or GPS. In various embodiments, the one or more communication devices communicate impact data to a medical provider (e.g., a doctor, a nurse, a caregiver, a team physiotherapist, a paramedic, a coach or a specially programmed remote server, such as a smartphone, a tablet, a remote computer, etc.). The medical provider can study the data and in response to the impact exceeding the predetermined threshold or the user showing signs of traumatic injury, communicate remotely to the processor via any of the communication devices to actuate delivery of the medication.

In various embodiments, a medical diagnostic and delivery device includes a mouth guard, a dental brace or a denture. The device can be positioned within a mouth or a buccal cavity of a user. A housing of the device can be shaped to be removably secured within the buccal cavity of the user, for example, include grooves shaped and sized to match a dental bite of the user. The device includes a cartridge containing one more substances or medications configured to be delivered to a user in the form a liquid, particles, or fine mist. Any suitable first air medication can be contained within the cartridge such as epinephrine, blood thinners (e.g., heparin, aspirin), painkillers (e.g., Tylenol®, non-steroidal anti-inflammatory drugs (NSAIDS), codine, morphine), b-vitamins or any other suitable drugs for limiting the extent of injury. In particular embodiments, the device can include a heating element to vaporize the substance and deliver the vapor to a user. The device includes a sensor, for example, an accelerometer for sensing, detecting or otherwise determining an impact on the head and/or any other body part of the user. The device also includes electronic circuitry that can include one or more of a power source, a specially programmed processor, a memory, a speaker, microphone and a display. The specially programmed processor may be configured to execute instructions for analyzing signals provided by the sensor and determining if the impact is beyond a critical or otherwise predetermined threshold. In various embodiments, in response to the impact exceeding the predetermined threshold, the processor commands the vaporizer to vaporize the medication contained within the cartridge so that the medication is delivered to the user. In other embodiments, the processor commands the injector to insert the needle into the user to deliver the medication. The electronic circuitry can also include communication and/or location devices such as Bluetooth®, Wi-Fi, RFID, cellular transceiver, and/or GPS. In various embodiments, the one or more communication devices communicate impact data to a medical provider (e.g., a doctor, a nurse, a caregiver, a team physiotherapist, a paramedic, a coach or a specially programmed remote server, such as a smartphone, a tablet, a remote computer, etc.). The medical provider can study the data and in response to the impact exceeding the predetermined threshold or the user showing signs of traumatic injury, communicate remotely to the processor via any of the communication devices to actuate delivery of the medication.

In various embodiments, a medical delivery device includes an airbag installed in the steering of a vehicle such as a car, a truck, a bus etc. In other embodiments, the airbag can include a side airbag or installed within a body of the vehicle, and/or a passenger side airbag. A medication cartridge is positioned within the airbag. The medication cartridge can contain any suitable medication such as epinephrine, blood thinners (e.g., heparin, aspirin), painkillers (e.g., Tylenol®, non-steroidal anti-inflammatory drugs (NSAIDS), codine, morphine), b-vitamins or any other suitable drugs for limiting the extent of injury. The vehicle includes an impact sensor, for example, an accelerometer or any other impact sensor commonly used in vehicles for impact detection. The impact sensor is coupled to a controller, for example, an on-board vehicle computer configured to interpret signals from the impact sensor and determine if the impact is above a predetermined threshold (e.g., greater than 2 G). In response to the impact being above a predetermined threshold, the airbag is deployed. The medication cartridge positioned within the airbag is configured such that deployment of the airbag causes the cartridge to burst and deliver the medication contained within the cartridge to the user. In various embodiments, the medication can be delivered as an aerosol or a mist to the user.

In various embodiments, a medical diagnostic and delivery device includes a face mask which can be worn by a doctor, a medical provider, an emergency medical technician (EMT), a nurse, a social worker or a volunteer care giver. The device includes a cartridge containing one more substance or medicaments configured to be delivered to a user in the form a liquid, particles, or fine mist. Any suitable first aid medication can be contained within the cartridge such as epinephrine, blood thinners (e.g., heparin, aspirin), painkillers (e.g., Tylenol®, non-steroidal anti-inflammatory drugs (NSAIDS), codine, morphine), b-vitamins, insulin, albuterol, fluticasone, anti-virals, anti-fungals, or any other or any other suitable first aid medication. The device includes a heating element to vaporize the substance and deliver the vapor to a user. The device includes a sensor for sensing, detecting or otherwise determining the presence of a pathogen (e.g., an air borne or blood borne pathogen) or a toxin (e.g., a toxic chemical) in the environment. In various embodiments, the sensor can include an electrochemical sensor, or a grid/matrix type sensor such as a lateral flow sensor, a paper or polymeric or any other suitable sensor. The device also includes electronic circuitry that can include one or more of a power source, a specially programmed processor, a memory, a speaker, microphone and a display. The specially programmed processor may be configured to execute instructions for analyzing signals provided by the sensor and determining if a pathogen or toxin is present in the environment or if the user has been exposed to the pathogen or toxin. In response to the pathogen or toxin present in the environment, the processor commands the vaporizer to vaporize the medication contained within the cartridge so that the medication is delivered to the user. The electronic circuitry can also include communication and/or location devices such as Bluetooth®, Wi-Fi, RFID, cellular transceiver, and/or GPS. In various embodiments, the one or more communication devices communicate sensor data to a medical provider (e.g., a doctor, a nurse, a caregiver, an EMT team or a specially programmed remote server, such as a smartphone, a tablet, a remote computer, etc.). The medical provider can study the data and in response to a possibility of the user being exposed to the pathogen or toxin, remotely actuate delivery of the medication to the user via instructions communicated to the processor via any of the communication devices included in the device. In various embodiments, the communication devices can also include an audio communication device, for example, a speaker to provide audible alerts to the user about possible exposure and medication delivery. In other embodiments, the communication devices can also include visual indicator, for example, an LED which can be visually observed by other user or personnel located in proximity of the user to provide an alert that the user is possibly exposed. In still other embodiments, the communication device can also include a speaker to allow the user to communicate orally with a caregiver or medical provider, for example, to update the caregiver on symptoms of the user.

In various embodiments, a medical diagnostic delivery device can include a vapor delivery device that can be accessed remotely by a caregiver or medical provider to diagnose a user and deliver one or more medications to the user. The device can include a housing including a face piece that can positioned over a mouth and/or a nose of the user. In other embodiments, the device can include a fluidic channel or tube which can be positioned on the lips of the user and configured to allow the user to inhale the medication vapors. The device includes cartridge containing one more substance or medicaments configured to be delivered to the user in the form of a liquid, particle, or fine mist. Any suitable first aid medication can be contained within the cartridge such as epinephrine, blood thinners (e.g., heparin, aspirin), painkillers (e.g., Tylenol®, non-steroidal anti-inflammatory drugs (NSAIDS), codine, morphine), b-vitamins, insulin, albuterol, fluticasone, anti-virals, anti-fungals, or any other or any other suitable first aid medication. In some embodiments, the cartridge can include a plurality of silos, each of which can contain a plurality of the same medication or different medications. In one embodiment, the cartridge includes a laterally displaceable cartridge that includes a plurality of substance silos arranged along a lateral axis of the cartridge. In another embodiment, the cartridge includes a circular cartridge including a plurality of substance silos positioned in radial orientation about a central axis of the cartridge. The circular cartridge is configured to be rotated about its central axis to position a predetermined substance for analysis or delivery to the user. The device includes a heating element to vaporize the substance and deliver the vapor to a user. The device also includes a sensor configured to receive a breath of user and sense, detect or otherwise determine one or more physical and/or biochemical parameters from the breath of the user. In various embodiments, the sensor can include an electrochemical sensor, or a grid/matrix type sensor such as a lateral flow sensor, a paper or polymeric sensor, a temperature sensor, a pulse sensor, an oxygen sensor, or blood pressure sensor or any other suitable sensor. The device also includes electronic circuitry that can include one or more of a power source, a specially programmed processor, a memory, a speaker, microphone and a display. The specially programmed processor may be configured to execute instructions for analyzing signals provided by the sensor and determining the one or more physical and/or biochemical parameter of the user. The electronic circuitry can also include communication and/or location devices such as Bluetooth®, Wi-Fi, RFID, cellular transceiver, and/or GPS. In various embodiments, a medical provider (e.g., a doctor, a nurse, a caregiver, an EMT team or a specially programmer remote server such as a smartphone, a tablet, a remote computer, etc.) can communicate a sensing request to the user, for example, indicating to the user that its time for performing a diagnostic or delivering medication, via the one or more communication devices. The processor and/or the medical provider can receive sensor data and analyze the data to determine a suitable medication contained within the cartridge for delivery to the user (e.g., in the form of vapor). Once the medication is delivered, the medical provider can communicate another sensing request to determine if the medication working. In response to the medication not working, the medical provider can request a different medication contained within the cartridge to be delivered to the user and then repeat the process.

In various embodiments, a medical diagnostic delivery device can include an epipen for delivering first aid medication to a user in response to a traumatic or non-traumatic emergency or medical emergency. The device can include an injector including a needle configured to deliver a medication to the user contained within a cartridge of the device. The cartridge can contain one more substance or medications configured to be delivered to a user in the form of a liquid or suspension. Any suitable first aid medication can be contained within the cartridge such as epinephrine, blood thinners (e.g., heparin, aspirin), painkillers (e.g., Tylenol®, non-steroidal anti-inflammatory drugs (NSAIDS), codine, morphine), b-vitamins, insulin, albuterol, fluticasone, anti-virals, anti-fungals, or any other or any other suitable first aid medication. In some embodiments, the needle can also be configured to draw a bodily fluid, for example, blood from the user after a first dose of the medication is delivered to the user. The bodily fluid can be communicated to a sensor which can be included in the device for sensing, detecting or otherwise determining one or more physical and/or biochemical parameters of the user. The device also includes electronic circuitry that can include one or more of a power source, a specially programmed processor, a memory, a speaker, a microphone and a display. The specially programmed processor may be configured to execute instructions for analyzing signals provided by the sensor and determining the one or more physical and/or biochemical parameter of the user. The processor can further be configured to determine from the parameters of the user if the medication is working. In instances where the medication does not work, the device can deliver another dose of the medication to the user and the process can be repeated. The electronic circuitry can also include communication and/or location devices such as Bluetooth®, Wi-Fi, RFID, cellular transceiver, and/or GPS. In various embodiments, the device can be configured to communicate a notification to an emergency medical provider and/or a caregiver concerning the injury or medical emergency.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The skilled artisan will understand that the drawings primarily are for illustrative purposes and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

FIG. 1A is a block diagram depicting an embodiment of a network environment comprising client devices in communication with server devices.

FIG. 1B is a block diagram depicting a cloud computing environment comprising client devices in communication with a cloud service provider.

FIGS. 1C and 1D are block diagrams depicting embodiments of computing devices useful in connection with the methods and systems described herein.

FIG. 2 is a side view of a medical diagnostic and delivery device that includes a protective head gear according to an embodiment.

FIG. 3 is a front view of another embodiment of medical diagnostic and delivery device configured to be positioned within a buccal cavity of a user.

FIG. 4 is a schematic flow diagram of a method of operating the devices of FIG. 2 or 3.

FIG. 5 is a front view of an embodiment of a medical delivery device that includes a medication disposed within an airbag of a vehicle.

FIG. 6 is a schematic flow diagram of a method of operating the medical device of FIG. 5.

FIG. 7 is a front view of another embodiment of a medical diagnostic and delivery device that includes a face mask.

FIG. 8 is a schematic flow diagram of a method of operating the medical device of FIG. 7.

FIG. 9 is a front view of a medical diagnostic and delivery device according to another embodiment.

FIGS. 10 and 11 are embodiments of cartridges that include a plurality of medication silos for containing one or more medication which can be included in the device of FIG. 9.

FIG. 12 is a schematic flow diagram of a method for operating the medical diagnostic and delivery device of FIG. 9.

FIG. 13 is a front view of yet another embodiment of medical diagnostic and delivery device.

FIG. 14 is a schematic flow diagram of a method for operating the medical diagnostic and delivery device of FIG. 13

The features and advantages of the inventive concepts disclosed herein will become more apparent from the detailed description set forth below when taken in conjunction with the drawings.

### DETAILED DESCRIPTION

Following below are more detailed descriptions of various concepts related to, and embodiments of, inventive variable counterweight systems and methods of operating variable counterweight systems. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes a network environment and computing environment which may be useful for practicing various computing related embodiments described herein.

Section B describes embodiments of systems and methods facilitating consumption of a substance through a vaporization and inhaler apparatus.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

### A. Computing and Network Environment

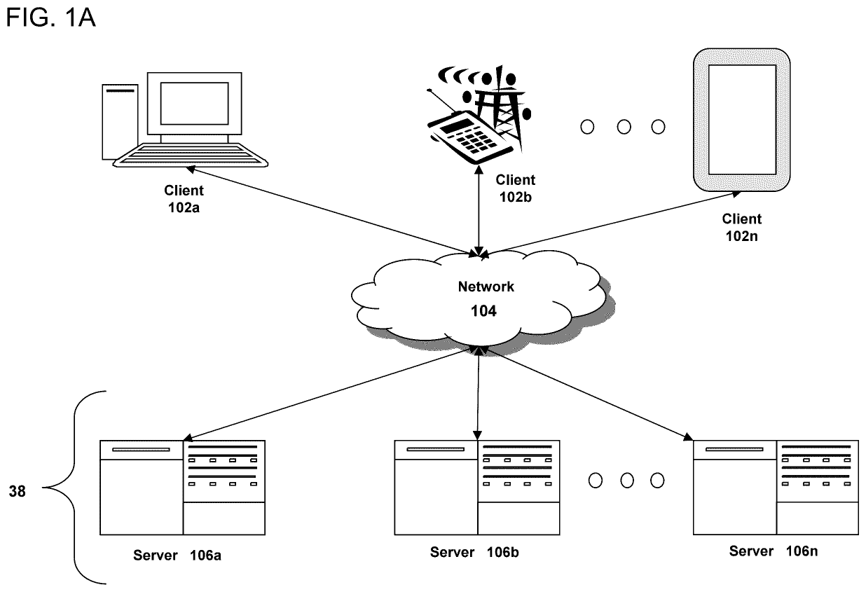

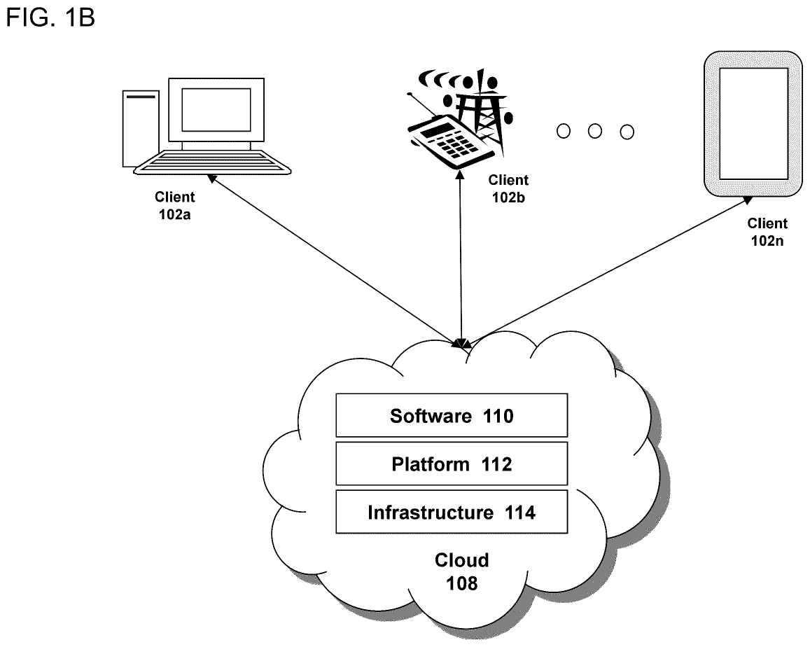

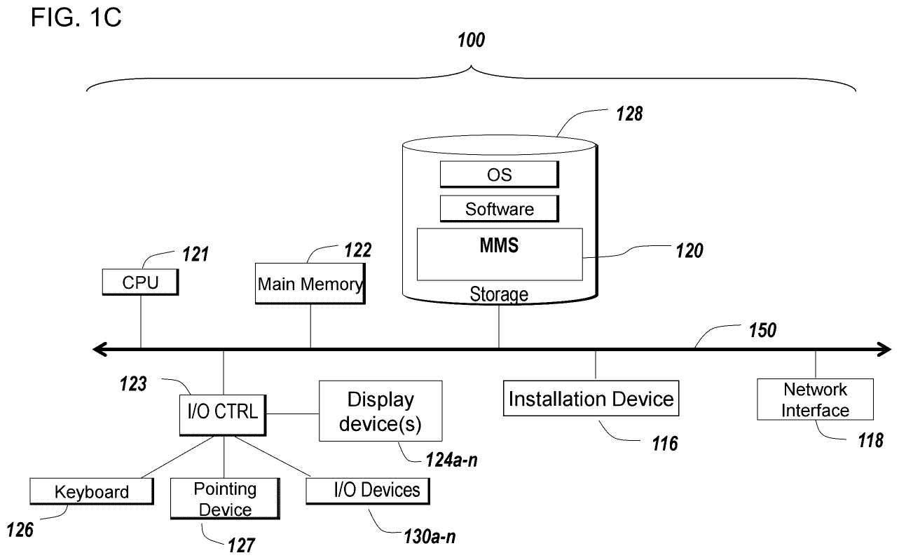

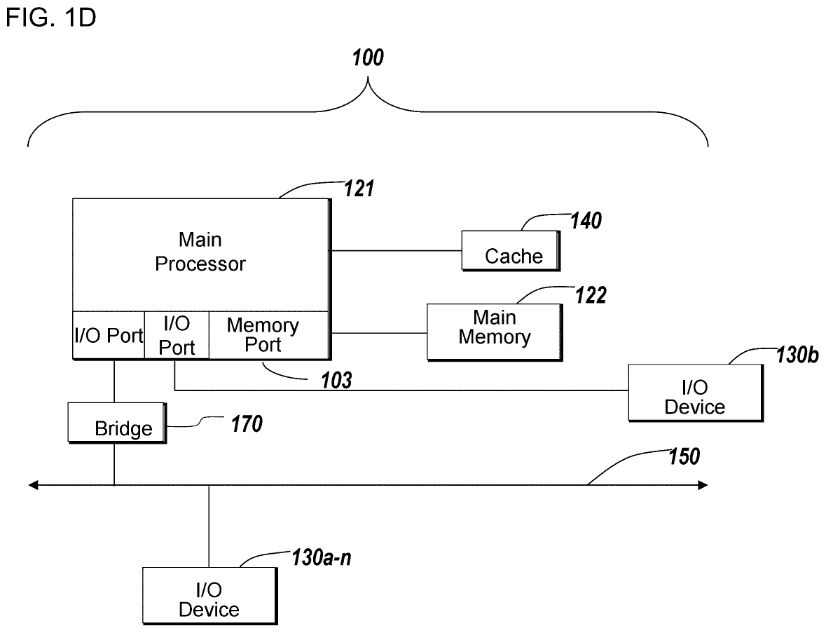

Prior to discussing specific inventive embodiments, it may be helpful to describe aspects of the operating environment as well as associated system components (e.g., hardware elements) in connection with the methods and systems described herein. Referring to FIG. 1A, an embodiment of a network environment is depicted. In brief overview, the illustrated exploring network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, NFC, RFID Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network, which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualized physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS include AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, and/or software of a medical diagnostic and/or delivery system 120 (e.g., the medical diagnostic and delivery device 200, 300, 500, 700, 900, 1300 or any other medical diagnostic device described herein). As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software 120 for the vaporization system. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call. In some embodiments, the communication device 102 is a wearable mobile computing device including but not limited to Google Glass and Samsung Gear.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the vaporization apparatus and related systems and methods disclosed herein.

### B. Systems and Methods of Administering Medications and Providing Medical Diagnosis Via Medical Diagnostic and Delivery Devices.

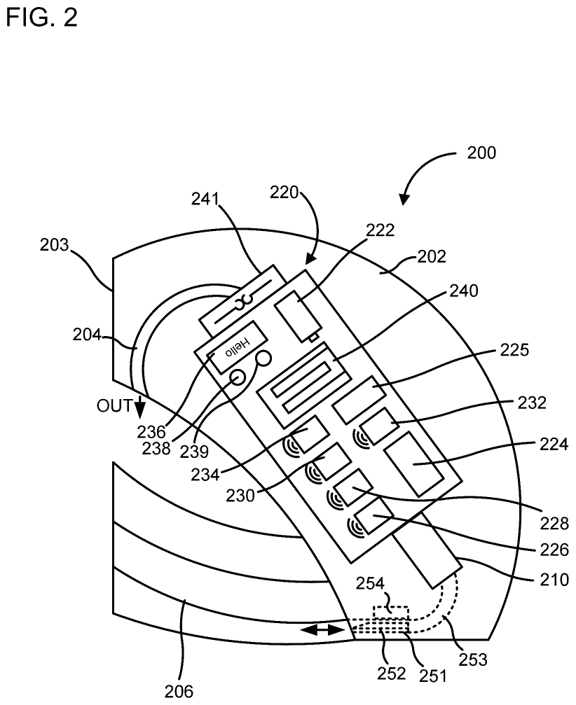

FIG. 2 is a front view of a medical diagnostic and delivery device 200 according to an embodiment. The device 200 includes a housing 202 defining an internal volume. FIG. 2 provides a partial cross-sectional view in the device 200. The housing 200 is shaped and sized to be a protective head gear that can be worn on the head of a user. Such a protective head gear can include a football helmet, an ice-hockey helmet, a cricket helmet, a hockey mask, a baseball referee mask, a ski helmet, a motorcycle helmet, a race car driver helmet, a construction worker helmet, or any other protective head gear. Foam polymers or other shock absorbing material can be positioned on an inner surface of the housing 202 to provide cushioning to the head of the user. As shown in FIG. 2 a front grill 206 is coupled to the housing to protect a face of the user. While shown as including the front grill 206, in other embodiments, the housing can include a portion surrounding a portion of the face of the user (e.g., a motor cycle helmet) and can also include a transparent protective shield (e.g., a Plexiglas or plastic shield) positioned proximal to a front end 203 of the housing 202.

The device 200 includes a cartridge 210 containing a medication to be delivered to a user through a fluidic channel 204 defined at the front end 203 with respect to a user. The cartridge 210 can include a liquid formulation or a solid formulation that may be crushed, pulverized, or atomized, or heated and vaporized for inhalation. The medication can be delivered to a user in response to an impact to the head of the user, or via remote activation by an overseer or a medical provider, as described herein. The medication may be delivered in the form of a liquid, a mist, powder, an aerosol or a vapor. Any suitable first aid medication can be contained within the cartridge 210 such as epinephrine, blood thinners (e.g., heparin, aspirin), painkillers (e.g., Tylenol®, non-steroidal anti-inflammatory drugs (NSAIDS), codine, morphine), b-vitamins or any other suitable drugs for limiting the extent of injury (e.g., brain damage) due to the impact injury. In various embodiments, the device 200 can also include a needle 252 defining a lumen 251 therewithin positioned within the housing 202. The lumen of the needle 252 can be in fluidic communication with the cartridge 210. An injector 254 (e.g., a lead screw, a plunger, a gas pump, a piezoresistive actuator) can be operatively coupled to the needle 252. In response to an impact exceeding a predetermined threshold, as described in further detail herein, the injector 254 can be configured to actuate the insertion of the needle into the user (e.g., into the back of the neck of the user) to intramuscularly or intravenously inject the medication into the user. In particular embodiments, a medication vapor can be produced if the impact is above a first predetermined threshold but below a second predetermined threshold, while the injection of the medication is performed if the impact is above the second predetermined threshold.

The cartridge 210 may include a cartridge identification code. The cartridge identification code may identify the substance (e.g., medication) contained in the cartridge, manufacturing date, manufacturer identification, batch/lot number, expiration information, or other pertinent information regarding the cartridge content. Furthermore, the housing 202 can be configured to provide temperature control to prevent degradation of the medication due to temperature fluctuations or exposure of the device 200 to extreme temperatures.

The device includes electronic circuitry 220 for performing various sensing, diagnostic and data communication functions as may be necessary. The electronic circuitry 220 includes a power source 222, for example a DC battery, a AA battery, a AAA battery, a coin cell, a kinetic power generation device, a solar panel or any other power source. A processor 224 is included in the electronic circuitry 220. The processor 224 is configured to execute instructions stored on a programmable memory 225 included in the electronic circuitry 220. The processor 224 is communicably coupled to a sensor 240 and configured to interpret signals provided by the sensor 240 included in the electronic circuitry 220. The sensor 240 can include an impact or force sensor such as, for example, an accelerometer configured to measure an impact force on the device 200. In some embodiments, the sensor 240 can also be configured to measure one or more physical parameters of the user such as temperature, blood oxygen, pulse rate, blood pressure, etc. The electronic circuitry 220 also includes a heater 241 configured to vaporize the medication for delivering to the user.

The signal from the sensor 240 is received an interpreted by the processor 224. The interpreting can include comparing the signals using algorithms, look up tables, sensor calibration information, noise reduction or data filtering using instructions stored on the programmable memory 225. Such parameters can include determining if the impact exceeds a critical or otherwise predetermined threshold (e.g., greater than 2 G). In various embodiments, the processor 224 can also be configured to determine if the impact force exceeds a first threshold (e.g., 2 G) and a second threshold (e.g., 4 G). In some embodiments, if the impact exceeds the first threshold but is below the second threshold, the processor 224 can command the heater 241 to vaporize the medication for delivering vapor to the user. Furthermore, in response to the impact exceeding the second threshold, the processor 224 can command the heater 241 to vaporize the medication as well as actuate the injector 254 for inserting the needle 252 into the user, thereby delivering liquid medication or intramuscularly or intravenously to the user.

In some embodiments, the electronic circuitry 220 can also include a counter to time the delivery of the substance (e.g., to determine a quantity delivered), an increment counter for each substance delivery suggestion, and/or to increment the counter each time a cartridge is changed. In various embodiments, the cartridge 210 can also include a cartridge identification code which may be electronically stored in the programmable memory 225. The electronic circuitry 220 also includes a plurality of communication device. For example, as shown in FIG. 2, the communication devices can include but are not limited to a Bluetooth® transceiver 226, a Wi-Fi transceiver 228, an RFID or NFC tag 230, and a cellular signal transceiver 232. The device 200 also includes GPS 234 for providing location information. A display 236, a speaker 238 and a microphone 239 are also provided. The display 236 and the speaker 238 can be configured to communicate visual and audio messages respectively to the user or a medical provider responding to the user after the trauma, such as, for example, power remaining, connectivity status, diagnosis information, cartridge ID, cartridge count, incorrect substance loaded, substance expired, substance contaminated, alerts, alarms, time, or any other beneficial information. The microphone 239 can be configured to allow oral input of commands to the device 200, for example, status inquiry, power on/power off, voice log, etc. In various embodiments, device 200 can include any combination of the communication devices described herein.

In particular embodiments, the device 200 is configured to communicate medication to the user wearing the device only in response to a remote command from an overseer or a medical provider (e.g., a doctor, a nurse, a caregiver, a team physiotherapist, a paramedic, a coach or a specially programmer remote server such as a smartphone, a tablet, a remote computer, etc.). For example, the processor can interpret the impact data obtained from the sensor 240 and communicate the sensor 240 data to the overseer or otherwise medical provider (e.g., on a smartphone app, a tablet app or a computer app possessed by the overseer or medical provider). The medical provider can analyze the data and/or recommendations provided by the processor, and responsive to the impact force exceeding the predetermined threshold (e.g., the first threshold and/or second threshold described herein) remotely activate delivery of the medication (e.g., medication vapor or liquid medication) via any of the communication device included in the electronic circuitry 220 (e.g., the Bluetooth® transceiver 226). In various embodiments, the overseer or medical provider can also communicate with the user via the speaker 238 and the microphone 239 included in the electronic circuitry 220. In still other embodiments, the sensor 240 can collect physical parameter data of the user to the overseer or medical provider (e.g., temperature, blood oxygen, pulse rate, blood pressure, etc.).

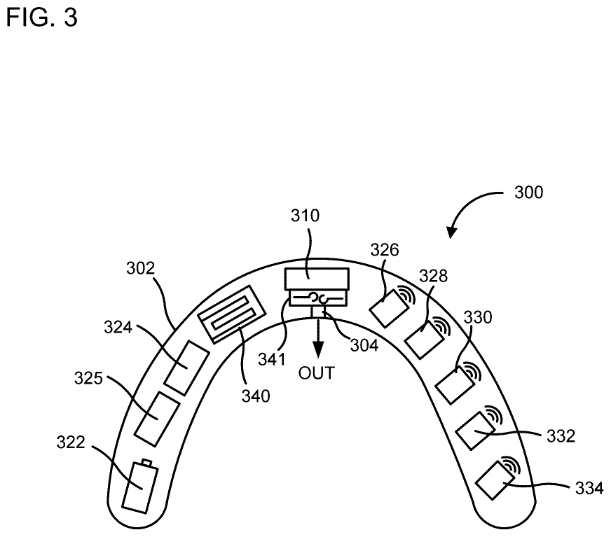

FIG. 3 is a schematic illustration of another embodiment of a medical diagnostic and delivery device 300. The device 300 includes a housing 302 defining an internal volume. The housing 302 has the form factor or otherwise shape of a dental retainer, dental brace or a mouth guard such that the device 300 can be positioned within the buccal cavity of a user and retained within the buccal cavity of the user. For example, the housing 302 can include a brace or clamps configured to clamp the teeth of the user and secure the housing around the teeth. In some embodiments, grooves, depressions or indents corresponding to the bite of the user can be defined on the housing 302, which allow the housing 302 to be removably positioned over the teeth of the user. A cartridge 310 is disposed within the internal volume and contains a medication therein, for example, any of the medications described with respect to the device 200. The housing 302 defines an outlet channel 304 in fluidic communication with at least one of a heater 341 included in the device 300 and the cartridge 310, and the buccal cavity of the user.

The device 300 includes a power source 322, for example, a battery such as a coin cell. In some embodiments, the power source 322 can include an electrochemical fuel cell configured to perform a redox reaction on saliva to generate electrical power. In other embodiments, the power source 322 can be configured to use kinetic power, for example, provided by movement of the jaw of the user during speaking or chewing. A memory 325 is provided that can store instructions executable by a processor 324 to perform the sensing and/or communication operations. The device 300 also includes one or more communication devices. For example, the device 300 can include a Bluetooth® transceiver 326, a Wi-Fi transceiver 328, an RFID or NFC tab 330, a cellular transceiver 332 and/or a GPS transceiver 334. In particular embodiments, the processor 324 can be configured to interpret signals from the Bluetooth® transceiver 326, or any of the other transceivers described herein, provided by a user. The instructions can include, for example, firmware updates, sensor 340 calibration parameters, device 300 on/off, or any other instructions. The instructions can be input into an app (e.g., a smartphone app, a tablet app, a computer app, or a program on remote server) which are communicated to the processor 324 and/or stored in the memory 325 via the Bluetooth® transceiver 326 or any of the other transceivers described herein. Similarly, the processor 324 can also be to communicate information to the user (e.g., to a smartphone app, a tablet app, a computer app, or a program on remote server) via the Bluetooth® transceiver. Such information can include, for example, raw sensor 340 data, processed data corresponding to impact on the head of the user, device 300 status (e.g., power remaining), sensor health data, or any other data providing overall information on diagnostic results and/or health of the user.

The device 300 includes a sensor 340 substantially similar to the sensor 240 described with respect to FIG. 2. The sensor 340 is configured to sense, detect or otherwise determine an impact on the head or any other body part of the user and produce an output signal which is communicated to the processor 324. The processor interprets the signal and responsive to the signal exceeding a predetermined threshold, commands the heater 341 to vaporize the medication producing medication vapors which are delivered to the user via the outlet channel 304. In other embodiments, the processor 324 can allow fluidic communication between the cartridge 310 and the outlet channel 304 responsive to the impact exceeding the predetermined threshold e.g., by opening a valve (not shown) to allow medication (e.g., liquid, powdered, suspension, aerosolized or a volatile medication) to be communicated from the cartridge 310 to the buccal cavity of the user. In such embodiments, the heater 341 can be excluded. In various implementations, a membrane can be positioned over an outlet of the outlet channel 304 to prevent clogging of the channel 304. In some embodiments, the power source 322 is activated to provide power to the electronic components included in the device 300 only when an impact is sensed by the sensor 340 to prevent unnecessary draining of the power of the power source 322. In still other embodiments, the impact data is communicated to an overseer or am medical provider (e.g., a doctor, a nurse, a caregiver, a team physiotherapist, a paramedic, a coach or a specially programmer remote server such as a smartphone, a tablet, a remote computer, etc.). The medical provider can analyze the data and if certain parameters are met, for example, the impact force exceeds a predetermined threshold or user is unresponsive, communicate a request to the processor 324 to deliver the medication to the user as described before herein with respect to the device 200.

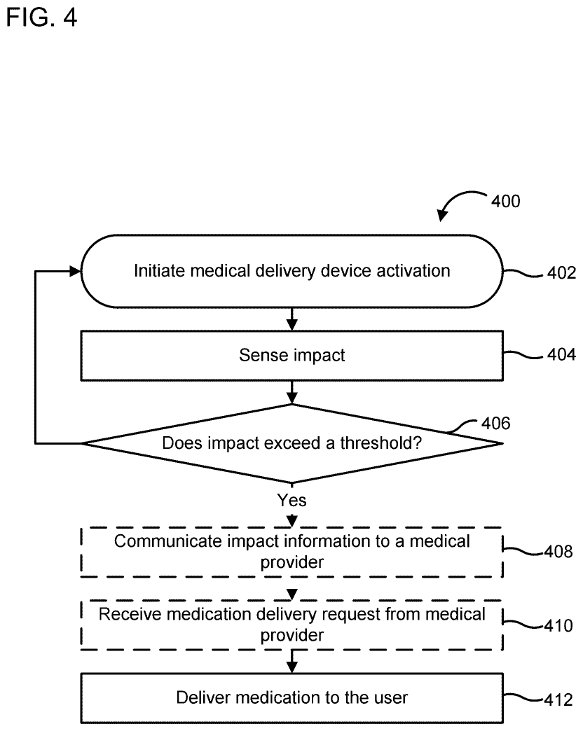

FIG. 4 is a schematic flow diagram of a method 400 for sensing, detecting or otherwise determining an impact force on a user using a medical diagnostic and delivery device, for example, the device 200 or 300, and deliver a medication to the user.

The method 400 includes initializing the medical device at 402, for example, commencing of athletic activity or sensing an impact on the user. The medical device can include the device 200 or 300. A device senses an impact, at 404. for example, the user suffers a sports injury, a vehicle crash, a fall or was involved in any other incident causing an impact on or near the head of the user. The sensing includes any of the sensing methodologies described herein which yield a signal (e.g., current, voltage, impedance, resistance, or optical signal) which is communicated to the processor in the form of a digital or analog signal. The device 402 determines if the impact exceeds a predetermined threshold at 406, for example, the impact is greater than 1 G, 2 G, 3 G, 4 G or any other predetermined value. In response to the impact exceeding the predetermined threshold, a the device delivers a medication to the user at 412, for example, the heater 241, 341 heats the medication contained within the cartridge 210, 310 to deliver a medication vapor to the user, or deliver a liquid, powdered, aerosolized medication to the user without vaporizing via the outlet channel 204, 304.

In various embodiments, the device communicates the impact information to a medical provider at 408 to determine if the impact exceeds the threshold, for example, via the Bluetooth® transceiver 226, 326 included in the device 200, 300 or any other communication device included in the device 200, 300. The medical provider analyzes the impact information, and communicates medication delivery request to the device 200, 300. The device receives the medication delivery request from the medical provider at 410, and the device at operation 412 delivers medication to the user based on the medication delivery request.

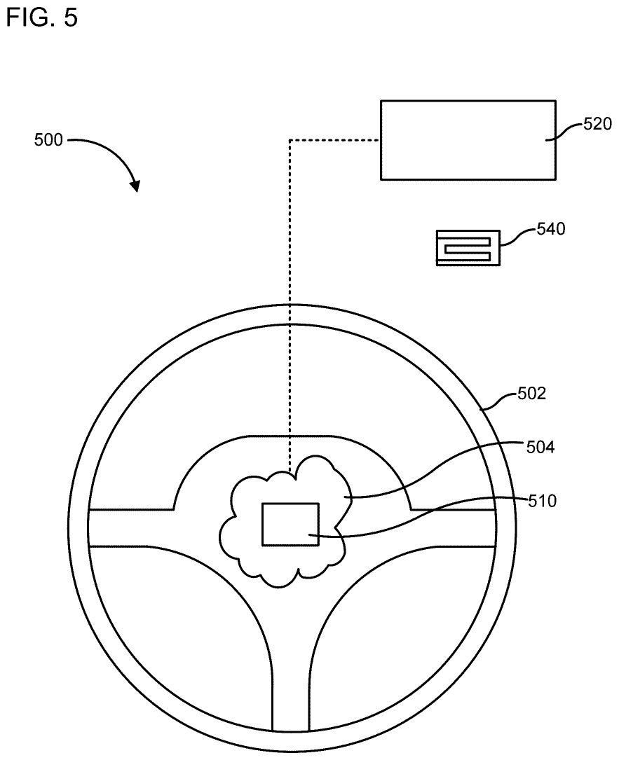

FIG. 5 is a schematic illustration of a medication delivery device 500 which is configured to deliver a substance, for example, a medication to driver, user, operator and/or passenger of a vehicle involved in an accident or a vehicle crash. The device 500 includes a medication cartridge 510 disposed within an airbag 504 positioned inside the steering wheel 502 of a vehicle. The vehicle includes an impact sensor 540 (e.g., an accelerometer) configured to sense, detect or otherwise determine an impact on the vehicle and provide impact data to a controller 520, for example, an onboard computer of the vehicle as is commonly known in the arts. The controller 520 is configured to analyze the impact data and deploy the airbag 504 if an impact force is greater than a predetermined threshold (e.g., 1 G, 2 G, 4 G, or any value therebetween or any other predetermined value).

The cartridge 510 includes one or more medications such as epinephrine, blood thinners (e.g., heparin, aspirin), painkillers (e.g., Tylenol®, non-steroidal anti-inflammatory drugs (NSAIDS), codine, morphine), b-vitamins disposed therein. The cartridge 510 is configured to explode, burst, tear open or otherwise release the medications contained therein when the airbag 504 is deployed. For example, the cartridge 510 can include a tearable pouch made from a tearable fabric, paper, plastic or any other suitable material to release the medication when the airbag 504 is deployed. The medication can be released in the form of a powder, a mist or an aerosol such that the medication can be inhaled by the user driving the vehicle and involved in the vehicle crash. In various embodiments, the medication can be contained directly in the airbag 504. For example, the medication can be disposed within the airbag 504 before the airbag 504 is installed in the vehicle. In other embodiments, the cartridge 510 can be removable positioned in the airbag 504 so that an old cartridge 510 (e.g., containing expired or degraded medication) can be replaced with a new medication cartridge 510 to ensure that fresh medication is always present in the airbag 504. For example, the cartridge replacement can be included in the regular maintenance schedule of the vehicle. In various embodiments, the medication can also be disposed in additional airbags of the vehicle, for example, side crash airbags, passenger side airbags, or any other airbags of the vehicle.

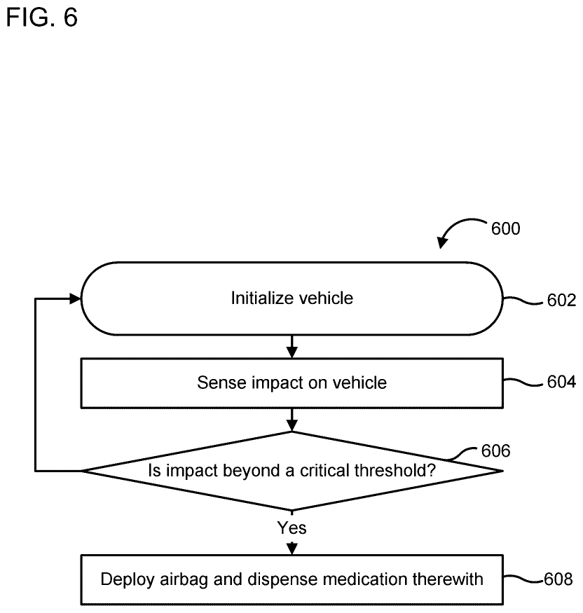

FIG. 6 is a schematic flow diagram of a method 600 for delivering medication contained within an airbag, for example, the medication contained within the cartridge 510 positioned within the airbag 504, to an operator or user of the vehicle involved in a vehicle crash or accident. The method 600 includes initializing the vehicle at 602, for example, starting the vehicle. The vehicles senses an impact on the vehicle at 604, for example, the sensor 540 which can be installed within a bumper or fender of the vehicle senses the impact on the bumper and generates an impact signal (e.g., a current or voltage corresponding to the impact force) which is communicated to the controller 520. The controller determines if the impact is beyond a critical threshold at 606, for example, the controller 520 interprets the signal from the sensor 540 and determines if the impact exceeded the critical threshold. If the impact force exceeds the critical threshold the airbag deploys dispensing the medication contained therein at 608. For example, the controller 520 analyzes the impact signal to determine if the impact is within or exceeds a critical threshold, and responsive to the impact exceeding the critical threshold, the airbag 504 is deployed urging the cartridge 510 to tear open or burst and release the medication contained therein, for consumption by the vehicle operator or user.

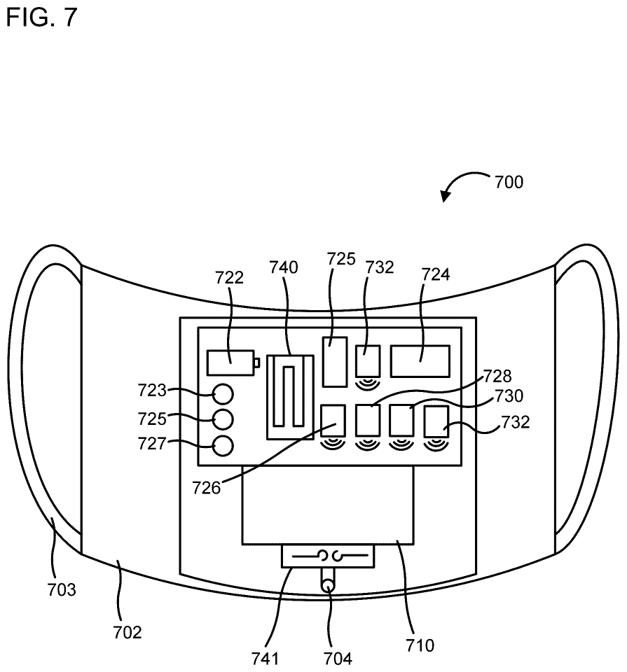

In various embodiments, a medical diagnostic and delivery device can include a face mask, such as a surgical mask configured to detect presence of toxins or pathogens in the environment and dispense medication to a user wearing the face mask to protect and/or treat the user. FIG. 7 is a schematic illustration of a medical diagnostic and delivery device 700. The device 700 includes a housing 702 having the form factor of a surgical mask or otherwise face mask (e.g., the face mask worn by surgeons, EMTs, aid workers, nurses, volunteers, social workers, etc.). Ear bands 703 or otherwise head bands are coupled to the housing and configured to be positioned around the ear or around the head of the user to secure the housing 702 over the nose and mouth of the user. The housing 702 is formed from a flexible material, for example, a fabric, a textile, a paper based material, a polymer or any other flexible material. Furthermore, the housing 702 is configured to prevent particulate matter from passing or crossing therethrough providing a first line of defense against air borne or fluid borne (e.g., blood borne, saliva borne etc.) toxins (e.g., harmful chemicals, toxic gases, etc.) or pathogens (e.g., air borne or blood borne viruses, bacteria, amoeba, etc.) which can be communicated to the user via the buccal cavity or nasal passage of the user.

The device 700, for example, a face mask is configured to sense, detect or otherwise determine the presence of air borne or blood borne pathogens or toxins in the environment and deliver a dose of medication to the user to provide first aid, prevent or limit spread of the toxin or pathogen, or provide the user with an energy boost to allow the user sufficient time to alert an emergency responder, or escape to a safe location free of the pathogen or environment. The device 700 includes a cartridge 710 containing a medication, for example, epinephrine, blood thinners (e.g., heparin, aspirin), painkillers (e.g., Tylenol®, non-steroidal anti-inflammatory drugs (NSAIDS), codine, morphine), b-vitamins, anti-virals, anti-bacterials or any other first aid medication contained therein. The cartridge 710 can include, for example, a flat pouch (e.g., a plastic pouch, a paper pouch, a vacuum sealed pouch, an aluminum line paper pouch, etc.) which is positioned between folds of the housing 702, and contains medication in any form, for example, liquid, powder, mist, aerosol, etc. The cartridge 710 is in fluidic communication with a heater 741 configured to heat and vaporize the medication which is delivered to a user via a fluidic channel 704 defined in the housing 702. In some embodiments, the vapors can penetrate through the housing 702 to be delivered to the user such that the fluidic channel 704 can be excluded.

The device 700 includes electronic circuitry 720 for performing various sensing, diagnostic and data communication functions as may be necessary. The electronic circuitry 720 includes a power source 722, for example a DC battery, a AA battery, a AAA battery, a coin cell, a kinetic power generation device, a solar panel or any other power source. A processor 724 is included in the electronic circuitry 720. The processor 224 is configured to execute instructions stored on a programmable memory 725 included in the electronic circuitry 720. The processor 724 is communicably coupled to a sensor 740 and configured to interpret signals provided by the sensor 740 included in the electronic circuitry 720, to determine if a pathogen or a toxin is present in the environment surround the device 700 (e.g., in the air or sprayed or splattered on the device 700). The interpreting can include comparing the signals using algorithms, look up tables, sensor calibration information, noise reduction or data filtering using instructions stored on the programmable memory 725.

The electronic circuitry 720 also includes a plurality of communication devices for receiving instruction from a user (e.g., input into an input interface (not shown) of the device 200, or communicated via a smart phone app, tablet app, remote server, etc.) or communicating information thereto. For example, as shown in FIG. 7, the communication devices can include but are not limited to a Bluetooth® transceiver 726, a Wi-Fi transceiver 728, an RFID or NFC tag 730, and a cellular signal transceiver 732. A visual indicator 723 (e.g., an LED light), a speaker 725 and a microphone 727 can also be provided. The communication devices can be configured to allow the user to communicate with other medical or service providers (e.g., using the speaker 725 and the microphone 727), for example, included in a team providing the medical service (e.g., a team of surgeons, aid workers, social workers, nurses, care givers), or with emergency service providers, for example, alert and communicate with 911 first responders, a dedicated emergency service provider team, or an overseers computer or remote server. The visual indicator 723 can be configured to emit a light corresponding to the presence of pathogens or toxins in the environment or provide other signals. In various embodiments, the visual indicator 723 can emit a light of a first color (e.g., green) to indicate that the device 700 is functioning properly, a blinking light (e.g., a blinking green light (to indicate low power, a blinking or solid light of a second color (e.g., yellow or orange to indicate the presence of a toxin, and a blinking or solid light of a third color (e.g., red) to indicate the presence of a pathogen in the environment. The electronic circuitry 720 can be flexible, for example, the components can be positioned on a flexible substrate such as a flexible plastic or embedded in a flexible polymer, to allow the device 700 to be comfortable worn on the face of the user.

The device 700 also includes GPS 734 for providing location information. In various embodiments, the medical device 700 can include any combination of the communication devices described herein. The sensor 740 is in fluidic communication with the environment, for example, through the housing 702 and configured to analyze the air around the housing 702 and/or any fluid splattered or sprayed on the housing 702 to sense, detect or otherwise determine the presence of one or more toxins or pathogens in the environment or the fluid contacting the housing 702. The sensor 740 can include a colorimetric sensor (e.g., fluorescence, bioluminescent or color producing sensor) which can produce an optical signal indicative of a value or level of the parameter. In such embodiments, the device 700 can include imaging or optical detection equipment communicably coupled to the processor 724 for interpreting the optical signal and transforming the signal into a digital or analog signal communicable to the processor 224.

In other embodiments, the sensor 740 can include an infrared sensor for analyzing the substance. In still another embodiment, the sensor 740 can include an electrochemical sensor configured to measure a redox current or voltage of the substance in the absence or presence of a catalyst (e.g., a natural or synthetic enzyme, a precious metal, etc.) which corresponds to the parameter of the substance. In another embodiment, the sensor 740 can include an impedance or conductance sensor or an electromagnetic sensor. In such embodiments, the sensor 2740 can include biological recognition molecules (e.g., polyclonal antibodies, monoclonal antibodies, antibody fragments, DNA, RNA, aptamers, synthetic recognition molecules and the likes) configured to bind with one or more components of the substance. The binding can alter an electrical or magnetic characteristic (e.g., impedance, conductance, dipole moment) of the sensor which can be interpreted by the processor 724 to determine the parameter. In still another embodiment, the sensor 740 includes a nanopore sensor.

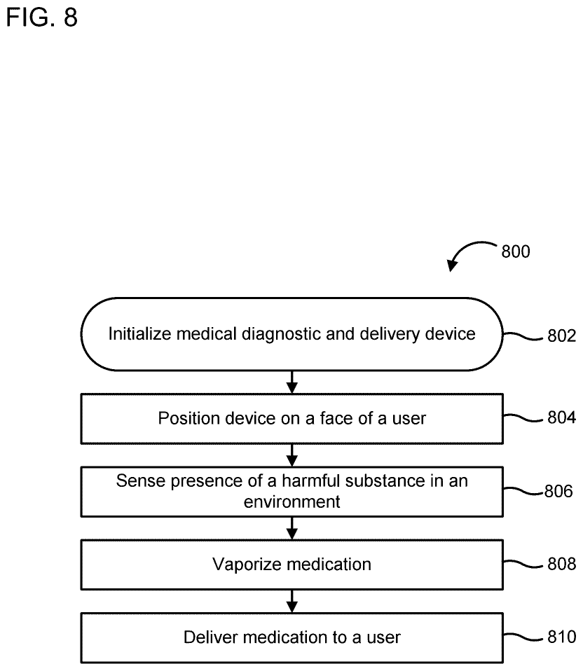

FIG. 8 is a schematic flow diagram of a method 700 for operating the device 700 described above herein. The method 700 includes initializing the medical diagnostic and delivery device at 802, for example, a user wears the device 700 over his face, activates the device 700 before or after wearing it (e.g., by actuating an on/off switch) or the presence of an environmental pathogen or toxin initializes the device 700. The device is positioned on the face of the user at 804, for example, over the nose and mouth of the user by positioning the bands 703 over the ears or tying the bands 703 around the head of the user so that the housing 702 is securely positioned over the nose and mouth of the user.

The device senses the presence of a harmful substance in an environment at 806, for example, the sensor 740 included in the device 700 senses, detects or otherwise determines the presence of a toxin or pathogen as described before herein in the air or in a fluid splattered or sprayed on the housing 702 of the device 700. The device vaporizes the medication at 808, for example, the processor 724 analyzes the sensor data and based on the determination that the toxin or pathogen is present, activates the heater 741 fluidly coupled to the cartridge 710 to vaporize the medication contained therewithin. The device delivers the medication to the user at 810, for example, the vaporized medication is communicated to the user via the fluidic channel 704 or through the housing 702 of the device 700. The fluidic channel 704 can be oriented towards an inner surface of the housing 702 of the device and configured to direct the medication vapor towards the nose or face of the user. In other embodiments, the outer surface of the housing 702 oriented away from the face of the user can be formed from a non-porous material, and the inner surface of the housing 702 oriented towards the face of the user can be porous. In this manner, the medication vapors can escape from the housing 702 through the porous inner surface of the housing 702 and are thereby, directed towards the nose and/or mouth of the user.

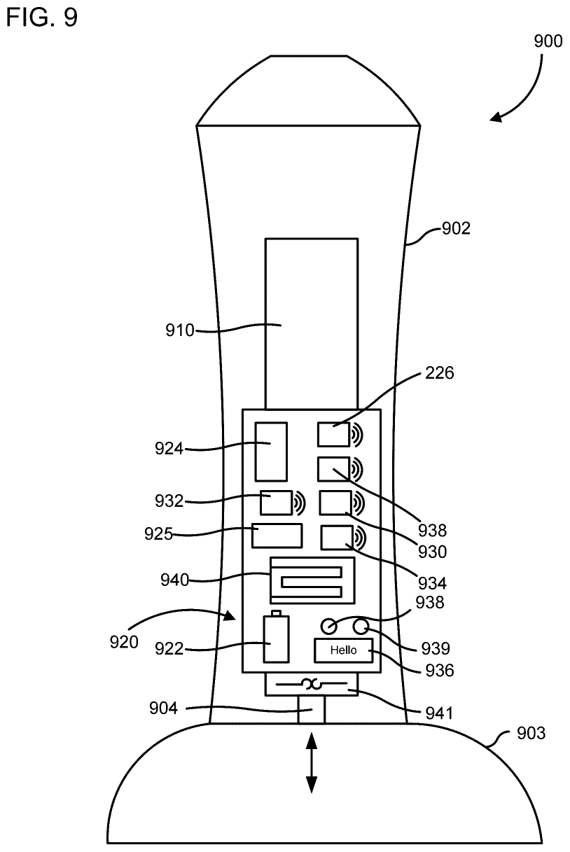

In some embodiments, any of the medical devices described herein can be configured to deliver a plurality of substances, for example, medications to a user and further be configured to be monitored by an overseer or medical provider to determine if the medication is working and remotely adjust a dosage and/or change medication delivered to the user. For example, FIG. 9 is a front view of a medical diagnostic and delivery device 900. The device 900 includes a housing 902 defining an internal volume. A face piece 903 is positioned at an end of the device 900 positionable over a mouth and/or nose of the user for delivering a substance contained within the cartridge 910 of the device via fluidic channel 904 which fluidly couples the cartridge 910 to the face piece 903. In various embodiments, the cartridge 910 is fluidly coupled to a heater 941 and configured to vaporize the substance so that a vapor of the substance (e.g., medication) is delivered to the user. In various embodiments, the fluidic channel 904 can also be configured to receive a breath of the user which is communicated to a sensor 940 included in the device 900. The sensor 940 can be configured to sense, detect or otherwise determine one or more physical or biochemical parameters of the user as an indicator of whether the medication delivered to the user is working. In other embodiments, a separate inlet channel (not shown) can also be defined within the housing 902 to deliver the breath to the sensor 940. In still other embodiments, a tube or nozzle can be fluidly coupled to the fluidic channel 904 in place of the face piece 903. A user can place the tube or otherwise nozzle on the lips of the user to inhale the vaporized medication or communicate breath into the device 900.

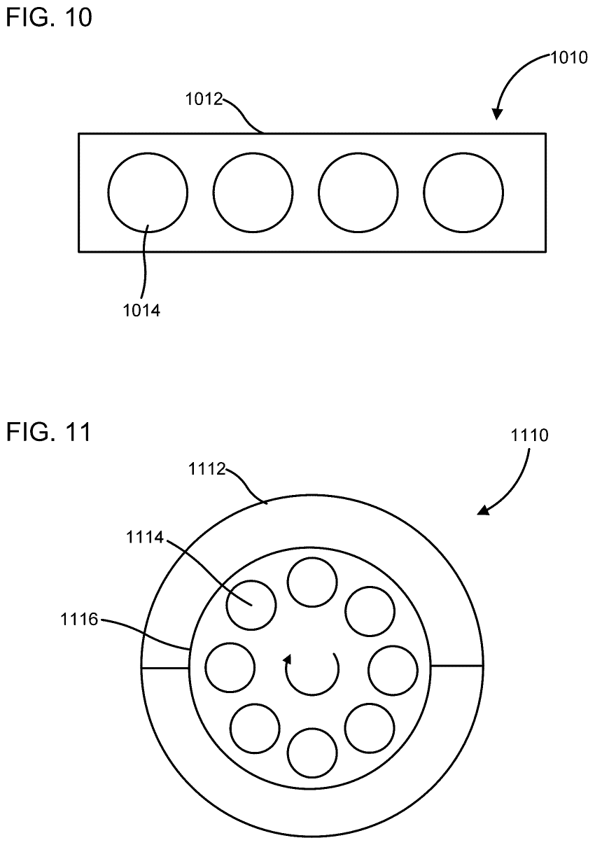

The cartridge 910 is positioned within the internal volume and contains one or more substances, for example, caffeine, panax, ginseng, gingko, biloba, bitter orange, cola-nut, guarana, natrum carbonicum, green tea, cocoa extract, cannabis, yerba mate, other vaporizable or inhalable supplements, pharmaceuticals, waxes, liquids, a breath alcohol masking agent, or medications such as epinephrine, blood thinners (e.g., heparin, aspirin), painkillers (e.g., Tylenol®, non-steroidal anti-inflammatory drugs (NSAIDS), codine, morphine), b-vitamins, insulin, albuterol, fluticasone, anti-virals, anti-fungals, or any other substance configured to provide therapeutic or medical relief to the user. In some embodiments, the cartridge 910 can be a repositionable cartridge configured to include a plurality of medications therewithin. For example, FIG. 10 is a front view of a cartridge 1010 that can be included in the device 1010 or any other medical device described herein. The cartridge includes a housing 1012 and a plurality of silos 1014 defined within the housing 1012. Each silo 1014 is filled or fillable with the same substance or different substances (e.g., different medications corresponding to a health management regimen of a user). The silos 1014 are defined along a longitudinal axis of the cartridge 1010. The cartridge 1010 is configured to be slidably repositioned within the device 1010 or any other medical device herein and moved laterally along the longitudinal axis to position any one of the silos 1014 in fluidic communication with the channel 1014 or the heater 1041, so that the substance present within the a particular silo 1014 is delivered to the user via the channel 904. The repositioning can be performed manually, or using a linear actuator (e.g., a lead screw or a plunger).

FIG. 11 shows another embodiment of a cartridge 1110. The cartridge 1110 includes a circular housing 1112 with a plurality of substance silos 1114 positioned radially about a central axis of the cartridge 1110. The cartridge 1110 can be rotatably repositioned within the device 900, for example manually, or by an actuator 1116, such as a servo motor which can be coupled to the cartridge 510.

Any of the cartridges 1010 or 1110 may be programed with identifying information that indicates what substances are in the cartridge and what silo position the substance is positioned at accordingly. The silos 1014 or 1114 may each have a unique position identifier and each cartridge may have a silo quantity identifier indicating the number and/or position of each silo. The information may be stored in a memory device of the cartridge 1010, 1110 and may be communicably retrieved or sent to the actuator to control the position of the cartridge in accordance with a user selection.

In particular embodiments, when cartridge 1110 rotates, as discussed further herein, it is possible for one sub-cartridge (e.g. silo 1112) to have the substance contained therein vaporized at a certain wattage/voltage, while a different sub-cartridge, for example containing a different substance, may be vaporized at a different wattage/voltage. Additionally one sub-cartridge might just dispense (and not vaporize) a substance @ill, powder, liquid, gel) or any combination of the above.

In particular embodiments each of the cartridges 1010, 1110 and the silos 1014, 1114, for example a cartridge containing any of the substances described herein, operates in a manner similar to an oven. In particular embodiments containing a liquid each of the cartridges 1010, 1110 and/or the silos 1014, 1114 use a wick/coil system. In particular embodiments the cartridges 1010, 1110 and/or the silos 1014, 1114 may use one or more of an ultrasonic diffuser, a cold air diffuser, an evaporative diffuser, or a heat diffuser. The ultrasonic diffuser uses electronic frequencies to create vibrations that are carried to the surface where oils are floating. The vibrations from the ultrasonic diffuser vaporize the oil and disperse it into the air without using any kind of heat. The cold air diffuser uses room-temperature air to blow the oil into a nebulizer where it is vaporized. The cold air diffuser can diffuse quickly and efficiently. The evaporative diffuser includes a fan that blows air through a pad or filter where the oil sits and vaporizes the oil on the pad. The heat diffuser uses a heat source to disperse the essential oil. In example embodiments, the cartridge 1010, 1110 may remain stationary and the actuator may reposition the heater for heating the silo 1014, 1114 and/or may reposition an output port to release the vaporized substance.

Referring back to FIG. 9, the substance cartridge 910 is in fluidic communication with at least one of the heater 941 (e.g., to allow vapor to be produced which is communicated to the user via the outlet channel) and the outlet channel 904 (e.g., to allow liquid substance, powder, a mist or aerosol to directly delivered to the user). The device 900 includes electronic circuitry 920 that includes a battery 922, a processor 924, a memory 925, the sensor 940 and the heater 941. The electronic circuitry 920 also includes communication devices including a Bluetooth® transceiver 926, a Wi-Fi transceiver 928, an RFID or NFC tag 930, a cellular transceiver 932 and a GPS transceiver 934. The device 900 can also include a display 936 to allow communication of visual cues, message and/or alerts to the user, for example, battery 922 power remaining, connectivity status, time, dosage information, dosage reminder, health status or any other visual communication to the user. The device 900 also includes a speaker 938, for example, to communicate audio alerts or messages to the user, as well as a microphone 939 to allow oral input of commands to the device 900. In various embodiments, the speaker 938 and microphone 939 can allow the user to communicate with an overseer or medical provider (e.g., a doctor, a nurse, a pharmacist, a caregiver, a smartphone or tablet app, a computer app or a remote server), for example, to discuss physical symptoms or obtain instructions on dosage.

As described before, the device 900 is configured to detect one or more physical or biochemical parameter of the user by analyzing a breath of the user. The user can blow into the fluidic channel 904 and the user's breath is communicated to the sensor 940. The sensor 940 can be substantially similar to the sensor 740 described before herein and configured to determine one or more physical parameters of the user such as, for example, glucose level, blood alcohol level (BAC), THC level (biomarker for marijuana), adrenal conditions (e.g., Cushing's disease, Addison's disease), hormone levels, altered female hormone states (e.g., PCOS, menopause, anovulation, pregnancy, irregular period cycles), altered male hormone states (e.g., hypogonadism, andropause, hyperestrogenic states), metabolic disorders (e.g., insulin resistance, diabetes, muscular dystrophy) benign and metastatic neoplasms (e.g., breast cancer, pancreatic cancer, prostate cancer, oral cancer, lung and throat cancer, etc.), infection diseases (e.g., HIV, viral hepatitis, flu, H1N1 flu, SARS virus, amoebiasis, heliobacter pylori infections, C. difficius infections, strep throat), food allergy, cortisol levels as indicators of stress, progesterone, or any other disease or medical condition for a which a biomarker is expressed in the breath of the user. While shown as included in the electronic circuitry 920, in particular embodiments, the sensor can be included in the cartridge 910.

In various embodiments, the sensor 940 can be a removable and/or replaceable sensor. In some embodiments, the sensor 940 is included in the cartridge 910 and replaced with the cartridge, for example, when the cartridge is expired. In other embodiments, the sensor 940 can be removably inserted or otherwise positioned in the device 900 and is configured to be replaced with a fresh sensor after a predetermined number of uses.

The device 900 can be configured to receive instructions from an overseer or medical provider to deliver a predetermined medication or substance included in the cartridge 910 to the user. Once the substance or medication has been delivered, the user can blow in the fluidic channel so that the sensor 940 senses, detects or otherwise determines one or more physical or biochemical parameters of the user. The processor 924 can be configured to receive signals from the sensor 940 and determine the medical condition presented by the user, for example, using instruction such as look up tables, algorithms or calibration curves stored on the processor 924 or on the memory 925. The diagnosis or raw data obtained from the sensor 940 can be communicated to the overseer or medical provider via the Bluetooth® transceiver 926, the Wi-Fi transceiver 928 or any other communication device included in the device 900. The overseer or medical provider can determine if the medication is working, and based on the diagnosis, increase a dosage of the medication or change the medication being delivered to the user.

For example, in one instance the user is diabetic and insulin is delivered to the user. The breath of the user is analyzed after delivering the insulin to determine if the user's blood glucose levels are within a predefined or desired range. Based on the determination, the processor 924, or a remote overseer or otherwise medical provider can command the processor 924 via instructions communicated through any of the communication devices included in the device 900 to take no further action, increase the dosage, change the medication or alert an emergency first aid responder.

As described before, the cartridge 910 can include a positionable cartridge (e.g., the cartridge 1010 or 1110) including a plurality of substances (e.g., medications) targeting a plurality of medical conditions, for example, any of the medical conditions described herein. Based on the diagnosis performed by the processor 924 determined from the parameter sensed by the sensor 940, the processor 924 can command an actuator (e.g., a linear actuator, a lead screw, a plunger or a servo motor) to reposition the cartridge 910 such that the identified substance which is to be delivered to the user is in fluidic communication with the outlet channel 904 or the heater 941. In particular embodiments, the cartridge 910 can be manually repositionable. In such embodiments, the processor 924 can send a command to a communication system (e.g., a display, an audible alarm via a speaker, or communicate to an app on smartphone, tablet or computer via Bluetooth®, Wi-Fi, cellular network, RFID or the likes) to indicate to the user the correct position of the cartridge 910. The user can then manually reposition the cartridge 910 to bring the identified substance in fluidic communication with at least one of the outlet channel 904 and the heater 941.

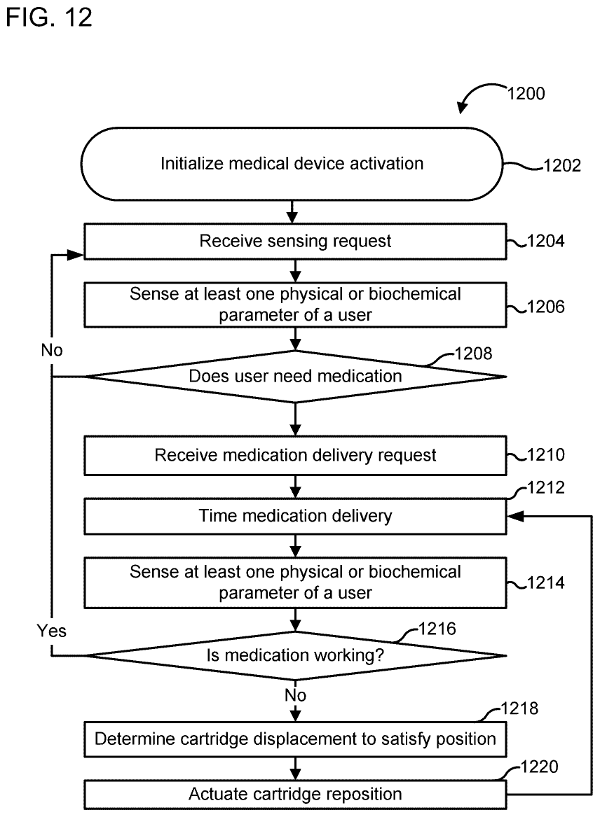

FIG. 12 is a schematic flow diagram of a method 1200 of operating the device 900. The method includes initializing medical device activation at 1202, for example in response to a user turning the apparatus 900 on, picking the device up, requesting a vapor draw, or inserting a new or different cartridge 910. The medical diagnostic device 900 can be calibrated after insertion based on a cartridge ID and the information provided by the cartridge (e.g., the cartridge 1010 or 1110 regarding the device silos 1014 or 1114, the number of silos, the position of the silos, and the contents of the silos).

The device receives a sensing request at 1204, for example, the user blows in the device 900, the user initiates an oral request via speaker 938, the user inputs a sensing command via the display 936, and/or a medical provider communicates a sensing request via any one of the communication devices included in the device 900. The device senses at least one physical and/or biochemical parameter of the user at 1206, for example, the sensor 940 senses, detects or otherwise determines one or more physical and/or biochemical parameters of the users breath. The device determines if the user needs medication at 1208, for example, the processor 924 interprets signals from the sensor 940 to determine if the user needs medication or the sensor 940 data is communicated to a remote overseer or medical provider to determine if the user needs the medication.

If no medication is needed, the method 1200 returns to operation 1204. If medication is required, the device receives a medication delivery request at 1210, for example, the processor 924 or the remote overseer or otherwise medical provider communicates a medication delivery request to the device 900. The device times the medication delivery at 1212, for example to determine an amount or dose of vaporized medication delivered to the user. The device again senses at least one physical or biochemical parameter of the user 1214, for example, after the medication is delivered, the user again blows into the face piece 903 of the device 900 to communicate breath to the sensor 940 via the fluidic channel 904 for analysis as described before herein. The device determines if the medication is working at 1216, for example, the substance or medication delivered to the user has succeeded in addressing a medical need of the user, for example, lowered or raised the level or value of the sensed physical or biochemical parameter (e.g., glucose level) to within a desired range.

If the medication is working, the method 1200 returns to operation 1204. If the medication is not working, the device determines a cartridge displacement to satisfy position at 1218, for example, if a first medication delivered to the user is not working, the processor 924 or a medical provider determines another medication present in the cartridge (e.g., the cartridge 910, 1010 or 1112) can possibly provide the desired therapeutic impact. Based on this determination, the cartridge displacement request is communicated to the device 900. The device actuates a cartridge reposition at 1220, for example, the cartridge is repositioned to align another silo of the cartridge (e.g., the cartridge 1010 or 1110) with the heater 941 or fluidic channel 904 such that different medication can now be delivered to the user. The cartridge repositioning can include slidably displacing the cartridge (e.g., the cartridge 1010) or revolving the cartridge (e.g., the cartridge 1110) manually or using an actuator (e.g., a linear actuator or a servo motor). The cartridge reposition can be determined by the processor 924 or communicated to the device 900 by the overseer or medical provider as described before herein. Once the cartridge is repositioned, or the cartridge was correctly positioned, the method 1200 returns to operation 1212 to deliver the different substance or medication to the user.

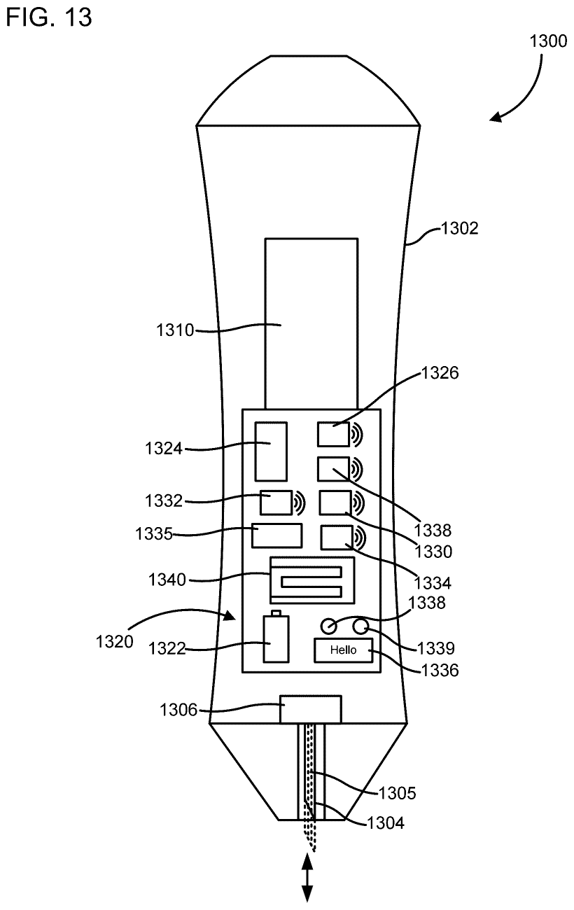

In various embodiments, a medication diagnostic and delivery device can be configured to resemble an epipen. For example, FIG. 13 is a front view of a medical diagnostic and delivery device 1300. A needle 1304 defining a lumen 1305 is positioned at one end of the device 1300. The needle 1304 is configured to be inserted into the body of a user to deliver a substance medication contained within a cartridge 1310 of the device 1300 to the user. For example, the cartridge 1310 can contain a first aid medication e.g., epinephrine or insulin to be delivered to the user. The device 1300 can also include an injector assembly 1306 configured to insert the needle 1304 into the body of the user. The injector assembly 1306 can include a plunger, a gas pump, a lead screw, piezoresistive actuator or any other actuator configured to insert the needle 1304 into the user. In various embodiments, the needle 1304 can also be configured to draw blood (e.g., via capillary action or via a vacuum pump included in the device 1300) from the user which is communicated to a sensor 1340 included in the device 900. The sensor 1340 can be configured to sense, detect or otherwise determine one or more physical or biochemical parameters of the user as an indicator of whether the medication delivered to the user is working.

The device 1300 includes electronic circuitry 1320 that includes a battery 1322, a processor 1324, a memory 125 and the sensor 1340. The electronic circuitry 1320 also includes communication devices including a Bluetooth® transceiver 1326, a Wi-Fi transceiver 1328, an RFID or NFC tag 1330, a cellular transceiver 1332 and a GPS transceiver 1334. The device 900 can also include a display 1336 to configured to communicate of visual cues, messages or alerts, for example, step by step instructions on how to use the device 1300, battery power remaining, connectivity status, medication expiration status or dose remaining, or any other information. The device 1300 can also include a speaker 1338, for example, to communicate audio alerts or messages to the user (e.g., step by step instructions on using the device 1300), as well as a microphone 1339 to allow oral input of commands to the device 1300. In various embodiments, the speaker 1338 and microphone 1339 can allow the user to communicate with an overseer or medical provider (e.g., a doctor, a nurse, a pharmacist, a caregiver, a smartphone or tablet app, a computer app or a remote server), for example, to discuss physical symptoms or obtain instructions on usage.

As described before, the device 1300 can be configured to detect one or more physical or biochemical parameter of the user by analyzing a blood drawn onto sensor 1340 via the needle 1304. The sensor 940 can be substantially similar to the sensor 740 and/or 940 described before herein and configured to determine one or more physical parameters of the user such as, for example, glucose level, blood alcohol level (BAC), THC level (biomarker for marijuana), adrenal conditions (e.g., Cushing's disease, Addison's disease), hormone levels, altered female hormone states food allergy, cortisol levels as indicators of stress, progesterone, or any other disease or medical condition for a which a biomarker is expressed in the blood of the user. While shown as included in the electronic circuitry 1320, in particular embodiments, the sensor can be included in the cartridge 1310. In various embodiments, the device 1300 can be disposable device, for example, configured for one time use.

Once the substance or medication has been delivered to the user, the sensor 1340 can sense, detect or otherwise determine the one or more physical and/or biochemical parameter of the user to determine if the medication is working. If the medication is not working as expected, for example as determined by the processor 1324 or an overseer or otherwise medical provider, a second bolus or otherwise dose of the medication can be delivered to the user. For example, in one instance the user is diabetic and insulin is delivered to the user. The blood of the user is analyzed after delivering the insulin to determine if the user's blood glucose levels are within a predefined or desired range. Based on the determination, the processor 924, or a remote overseer or otherwise medical provider can command the processor 924 via instructions communicated through any of the communication devices included in the device 900 to take no further action, increase the dosage, change the medication or alert an emergency first aid responder. Similarly, in other embodiments, a person has allergies (e.g., food allergies, bee sting allergy, environmental allergy or any other allergy) and the device 1300 delivers epinephrine to the user. As described before, a first dose of the epinephrine is determined to the user and the device 1300 than determines if the epinephrine is working based on the one or more physical and/or biochemical parameters sensed in the blood of the user. If the epinephrine is not working as expected, a second dose of the epinephrine is delivered to the user. Furthermore, in various embodiments, the device 1300 can also be configured to alert one or more of an emergency medical service provider and/or a caregiver with pertinent information, for example, the device 1300 is used which alerts the medical provider of the emergency situation, dosage of medication delivered to the user, physical health of the user (e.g., as determine from the measured one or more physical and/or biochemical parameters) and/or physical location of the user.

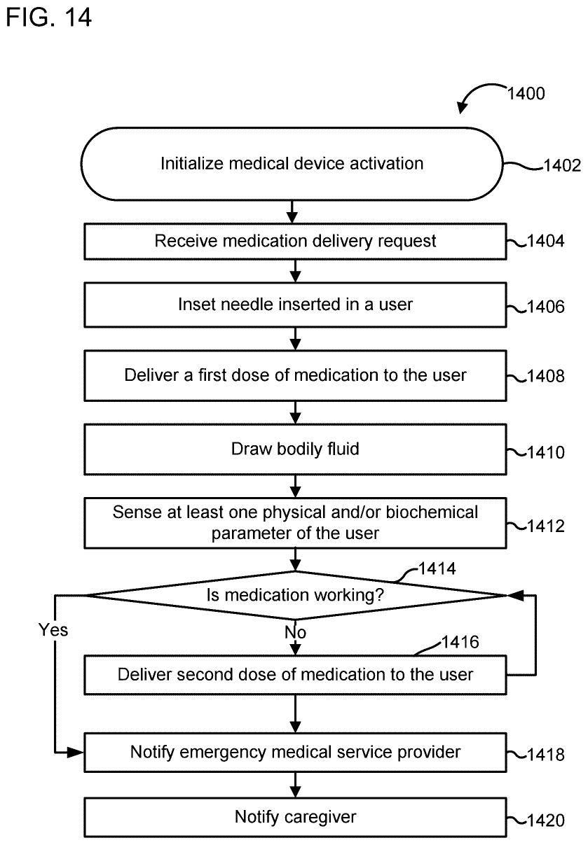

FIG. 14 is a schematic flow diagram of a method 1400 of operating the device 1300. The method 1400 includes initializing the medical device at 1402, for example, turning the device 1400 on by switching an on/off switch, actuating the needle 1304 or otherwise inserting the needle 1304 into the body of a user. The device receives a medication delivery request at 1404, for example, the injector assembly 1306 is activated. The device inserts a needle into the user at 1406, for example, an end of the device including the needle 1304 positioned therein is positioned adjacent to a body part (e.g., thigh, buttock, etc.) of the user and the injector assembly 1306 inserts the needle 1304 into the body part of the user. The device delivers a first dose of the medication to the user at 1408, for example, a dose of insulin or epinephrine is delivered to the user via the needle 1304.

The device draws a bodily fluid at 1410, for example, the needle 1304 draws blood from the user and delivers to the sensor 1340. The device senses a physical and/or biochemical parameter of the user at 1412, for example, the sensor 1340 senses, detects or otherwise determines one or more physical and/or biochemical parameter of the user from the user's blood (e.g., blood glucose level, blood oxygen level, quantity of an antibody and/or any other biomarker) which is indicative of the user's health or responsiveness to the medication. The device determines if the medication is working at 1414, for example, the physical and/or biochemical parameter is tending towards a normal range. If the medication is working, the device notifies an emergency medical service provider 1418, for example, an alert or signal can be communicated to 911, the local police or fire department, the local hospital or any other service provider. The notification can be communicated via any of the communication devices included in the device 1300. For example, a Bluetooth® signal can be communicated to a smartphone app installed on a smartphone of the user which then places a phone call with a prerecorded message to the emergency medical service provider. In other embodiments, the cellular transceiver 1332 can be used to place a call to the emergency medical service provider or otherwise notify the emergency medical service provider. The device also notifies a caregiver at 1420, for example, once the emergency medical service provider is notified, the device 1300 notifies the caregiver such as a relative, a next of kin, nurse or any other caregiver. If it is determined at operation 1414 that the medication is not working a second dose of the medication is delivered to user at 1416, and the method 1400 returns to operation 1414 thereafter.

In various embodiments, any of the medical diagnostic and delivery devices described herein can be tamper proof. This is to prevent medication abuse and fraud. In some implementations, the devices can include an alert system that triggers an alert upon determining that the device has been tampered or an attempt to tamper the device was made. In some implementations, the device can include one or more security modules. The security modules can include hardware and software to ensure that the medication is dispensed to an authorized user or patient. In some implementations, the one or more devices can include a user recognition or identification system. In some implementations, the one or more devices can include a fingerprint reader, an iris scanner, or any other biometric scanner to confirm the identity of the user. In some implementations, the devices can be password protected. In some implementations the devices can only be actuated via a second device, such as a smartphone or tablet, registered with the medical management system.

In various embodiments, any of the medical diagnostic and delivery devices described herein can include a temperature control module to control the temperature of medication within the device, for example disposed within a substance or medication cartridge positioned within the device. In this way, if the medicine in the device is to be maintained at a particular temperature, and the device is in a location that has an ambient temperature much higher than the temperature at which to maintain the medicine, the device can provide cooling to the medication. Conversely, the device can provide heating to medications that are supposed to be stored or maintained at temperatures higher than the ambient temperature at which the device is located.

In certain embodiments, the electronic circuitry included in each of the medical diagnostic devices 200, 300, 500, 700, 900, 1300 or any other device described herein can include a diagnostic control system. The diagnostic control system may include a controller structured to perform certain operations to cause sensing or delivery of a substance. The controller may be a single device or a distributed device, and the functions of the controller may be performed by hardware and/or as computer instructions on a non-transient computer readable storage medium.

In certain embodiments, the controller includes one or more modules structured to functionally execute the operations of the controller. In certain embodiments, the controller includes sensor modules configured to measure time lapse, energy consumption, product consumption, rotation position, a change in rotation, linear position, a change in a linear position, product location, product ingredients, or other diagnostic, delivery or vaporization system operating parameters or conditions impacting the use, sensing, dispensing, or operation of the medical diagnostic system.

The description herein including modules emphasizes the structural independence of the aspects of the controller, and illustrates one grouping of operations and responsibilities of the controller. Other groupings that execute similar overall operations are understood within the scope of the present application. Modules may be implemented in hardware and/or as computer instructions on a non-transient computer readable storage medium, and modules may be distributed across various hardware or computer based components.

Example and non-limiting module implementation elements include sensors providing any value determined herein, sensors providing any value that is a precursor to a value determined herein, datalink and/or network hardware including communication chips, oscillating crystals, communication links, cables, twisted pair wiring, coaxial wiring, shielded wiring, transmitters, receivers, and/or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), and/or digital control elements.

Non-limiting examples of various embodiments are disclosed herein. Features from one embodiments disclosed herein may be combined with features of another embodiment disclosed herein as someone of ordinary skill in the art would understand.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It should be noted that the orientation of various elements may differ according to other exemplary embodiments, and that such variations are intended to be encompassed by the present disclosure. It is recognized that features of the disclosed embodiments can be incorporated into other disclosed embodiments.

It is important to note that the constructions and arrangements of apparatuses or the components thereof as shown in the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

While various inventive embodiments have been described and illustrated herein, those of ordinary skill in the art will readily envision a variety of other mechanisms and/or structures for performing the function and/or obtaining the results and/or one or more of the advantages described herein, and each of such variations and/or modifications is deemed to be within the scope of the inventive embodiments described herein. More generally, those skilled in the art will readily appreciate that, unless otherwise noted, any parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way unless otherwise specifically noted. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. A medical diagnostic and delivery device comprising:
a housing configured to be removably secured within a buccal cavity of a user; and
a cartridge including medication configured to be delivered to the user via the housing.
